(19) United States
(12) Patent Application Publication
(10) Pub. No.: US 2024/0074674 A1
(43) Pub. Date: Mar. 7, 2024

(54) AEROSOL QUANTITY ESTIMATION SYSTEM, AEROSOL QUANTITY ESTIMATION METHOD, AND NON-TRANSITORY COMPUTER-READABLE RECORDING MEDIUM

(71) Applicant: Panasonic Intellectual Property Management Co., Ltd., Osaka (JP)

(72) Inventors: TATSUSHI OHYAMA, Gifu (JP); MARIKO MIYASHITA, Hyogo (JP)

(21) Appl. No.: 18/503,467

(22) Filed: Nov. 7, 2023

Related U.S. Application Data
(63) Continuation of application No. PCT/JP2022/019579, filed on May 6, 2022.

(30) Foreign Application Priority Data
May 21, 2021 (JP) 2021-085766

(51) Int. Cl. A61B 5/091; G10L 15/10

(52) U.S. Cl. CPC A61B 5/091; G10L 15/10

(57) ABSTRACT

An aerosol quantity estimation system includes a detector that detects a voice and a controller that estimates a number of aerosol particles released to a space where an utterer who has emitted the voice detected by the detector exists from a certain speech sound included in the voice on a basis of a correlation between the certain speech sound and a number of aerosol particles released from the utterer when the utterer utters the certain speech sound. An aerosol quantity estimation method includes detecting a voice and estimating a number of aerosol particles released to a space where an utterer who has emitted the detected voice exists from a certain speech sound included in the voice on a basis of a correlation between the certain speech sound and a number of aerosol particles released from the utterer when the utterer utters the certain speech sound.

### BACKGROUND

### 1. Technical Field

The present disclosure relates to an aerosol quantity estimation system, an aerosol quantity estimation method, and a non-transitory computer-readable recording medium.

### 2. Description of the Related Art

Japanese Unexamined Patent Application Publication No. 2011-174624 discloses a method for detecting occurrence of coughing in a room and a position at which the coughing has occurred on the basis of a sound in the room. Japanese Unexamined Patent Application Publication No. 2020-030010 discloses a ventilation apparatus that detects occurrence of coughing or sneezing on the basis of a sound and that performs ventilation after a certain period of wait time.

S. Asadi et al., “Aerosol emission and superemission during human speech increase with voice loudness”, Nature Scientific Reports, February 2019, Vol. 9, No. 1 describes that there is a correlation between loudness of a voice and the number of aerosol particles released. Philip Anfinrud et al., “Visualizing Speech-Generated Oral Fluid Droplets with Laser Light Scattering”, N Engl J Med 2020; 382:2061-2063 describes that in a case where a subject says, “Stay healthy”, a large number of aerosol particles is released when “th” is pronounced.

### SUMMARY

With the above examples of the related art, however, it is difficult to accurately estimate the number of aerosol particles caused by an utterer when the utterer has uttered speech sounds.

One non-limiting and exemplary embodiment provides an aerosol quantity estimation system, an aerosol quantity estimation method, and a non-transitory computer-readable recording medium capable of accurately estimating the number of aerosol particles caused by an utterer when the utterer has uttered speech sounds.

In one general aspect, the techniques disclosed here feature an aerosol quantity estimation system including a detector that detects a voice and a controller that estimates a number of aerosol particles released to a space where an utterer who has emitted the voice detected by the detector exists from a certain speech sound included in the voice on a basis of a correlation between the certain speech sound and a number of aerosol particles released from the utterer when the utterer utters the certain speech sound.

With the aerosol quantity estimation system according to the aspect of the present disclosure and the like, the number of aerosol particles caused by an utterer when the utterer has uttered speech sounds can be accurately estimated.

It should be noted that these general or specific aspects may be implemented as an apparatus, a system, a method, an integrated circuit, a computer program, a computer-readable storage medium, or any selective combination thereof. The computer-readable storage medium includes, for example, a nonvolatile storage medium such as a compact disc read-only memory (CD-ROM).

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a diagram illustrating a result of measurement of sound pressure dependence of the number of aerosol particles released as a result of a single cough by a person;

FIG. 2 is a diagram illustrating a result of measurement of sound pressure dependence of the number of aerosol particles released when monosyllables including all basic sounds, dakuten sounds, and handakuten sounds are uttered once;

FIG. 3 is a diagram illustrating a result of measurement of sound pressure dependence of the number of aerosol particles released when a monosyllable “shi” (Japanese) is uttered once;

FIG. 4 is a diagram illustrating a result of measurement of sound pressure dependence of the number of aerosol particles released when a monosyllable “su” (Japanese) is uttered once;

FIG. 5 is a diagram illustrating a result of measurement of sound pressure dependence of the number of aerosol particles released when a monosyllable “chi” (Japanese) is uttered once;

FIG. 6 is a diagram illustrating a result of measurement of sound pressure dependence of the number of aerosol particles released when a monosyllable “tsu” (Japanese) is uttered once;

FIG. 7 is a diagram illustrating a result of measurement of sound pressure dependence of the number of aerosol particles released when monosyllables of vowels are uttered once;

FIG. 8 is a diagram illustrating a result of measurement of sound pressure dependence of the number of aerosol particles released when monosyllables “ra” (Japanese), “ri” (Japanese), “ro” (Japanese), “zo” (Japanese), “da” (Japanese), “ba” (Japanese), and “pa” (Japanese) are uttered once;

FIG. 9 is a block diagram illustrating an example of the configuration of an aerosol quantity estimation system according to a first embodiment;

FIG. 10 is a diagram illustrating a result of measurement of sound pressure dependence of the number of aerosol particles released when a monosyllable “ki” (Japanese) is uttered once;

FIG. 11 is a diagram illustrating a result of measurement of sound pressure dependence of the number of aerosol particles released when a monosyllable “na” (Japanese) is uttered once;

FIG. 12 is a diagram illustrating a result of measurement of sound pressure dependence of the number of aerosol particles released when a monosyllable “to” (Japanese) is uttered once;

FIG. 13 is a diagram illustrating a result of measurement of sound pressure dependence of the number of aerosol particles released when a monosyllable “ni” (Japanese) is uttered once;

FIG. 14 is a table illustrating an example of certain speech sounds;

FIG. 15 is a flowchart illustrating an example of an operation performed by the aerosol quantity estimation system according to the first embodiment;

FIG. 16 is a diagram illustrating a specific example of a process performed by an aerosol quantity analysis portion;

FIG. 17 is a diagram illustrating a specific example of a process performed by a risk analysis section according to a modification of the first embodiment;

FIG. 18 is a block diagram illustrating an example of the configuration of an aerosol quantity estimation system according to a second embodiment;

FIG. 19 is a flowchart illustrating an example of an operation performed by the aerosol quantity estimation system according to the second embodiment; and

FIG. 20 is a diagram illustrating a specific example of a process performed by the aerosol quantity analysis portion.

### DETAILED DESCRIPTIONS

### Underlying Knowledge Forming Basis of Present Disclosure

Droplets are generally released from a person's mouth when he/she sneezes, coughs, or speaks. Droplet nuclei, which are approximately 10 μm or smaller in size, remain in air as aerosol after moisture in the droplets evaporates. The number of aerosol particles released as a result of a single sneeze is generally said to be about 40,000.

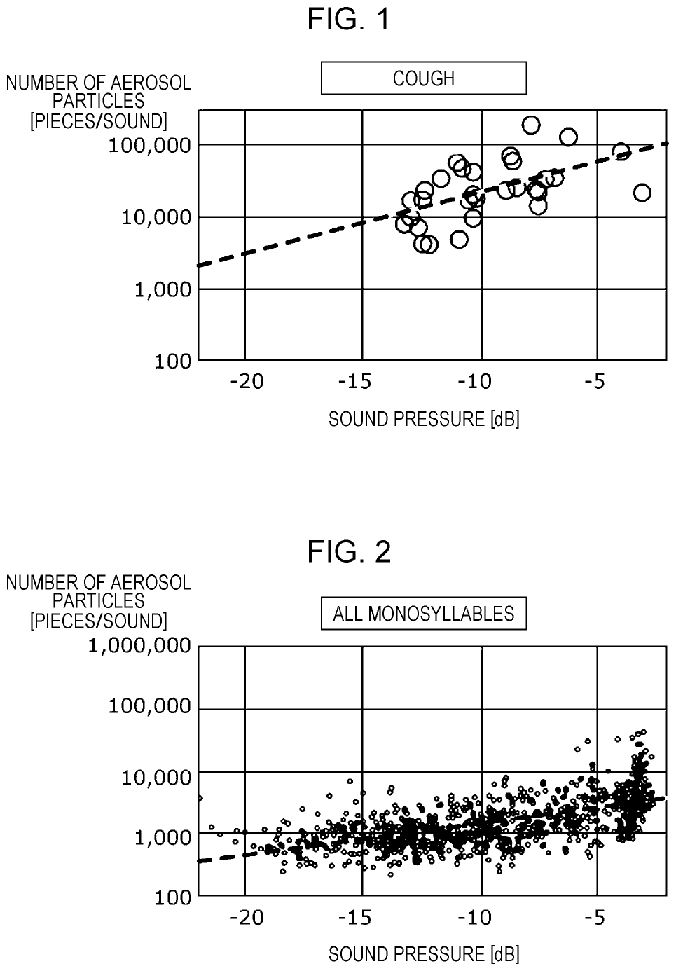

FIG. 1 is a diagram illustrating a result of measurement of sound pressure dependence of the number of aerosol particles released as a result of a single cough by a person. In FIG. 1, a horizontal axis represents sound pressure [dB], and a vertical axis represents, as logarithms, the number of aerosol particles released as a result of a single cough.

As illustrated in FIG. 1, the number of aerosol particles released as a result of a single cough is positively correlated with sound pressure. That is, in FIG. 1, the higher the sound pressure of a sound made by a cough, the larger the number of aerosol particles released from the mouth. More specifically, the number of aerosol particles released as a result of a single cough can be represented by an exponential function of sound pressure.

A broken line in FIG. 1 indicates an approximate exponential curve based on measurement points. The broken line indicates, for example, that 6,000 to 100,000 aerosol particles are released from the mouth depending on the sound pressure of a cough. Although the sound pressure of a cough has been described here, a similar trend is observed in the sound pressure of a sneeze and the number of aerosol particles.

The sound pressure is specifically a maximum value of amplitude of an audio signal obtained by detecting a voice emitted by an utterer using a microphone and is a relative value. The voice includes utterances or monosyllables. The utterances are emission of speech sounds. The speech sounds are sounds used as language and exclude reflex sounds caused by coughs, sneezes, and the like, which are nonverbal sounds. The monosyllables herein refer to speech sounds broken down to basic sounds (e.g., “shi” (Japanese)), dakuten sounds, handakuten sounds, or the like. Vowels are “a” (Japanese), “i” (Japanese), “u” (Japanese), “e” (Japanese), and “o” (Japanese) among the basic sounds.

FIG. 2 is a diagram illustrating a result of measurement of sound pressure dependence of the number of aerosol particles released when monosyllables including all the basic sounds, the dakuten sounds, and the handakuten sounds are uttered once. In FIG. 2, a horizontal axis represents sound pressure [dB], and a vertical axis represents, as logarithms, the number of aerosol particles released as a result of one utterance of the monosyllables. As illustrated in FIG. 2, monosyllables are roughly double-logarithmically proportional to the sound pressure.

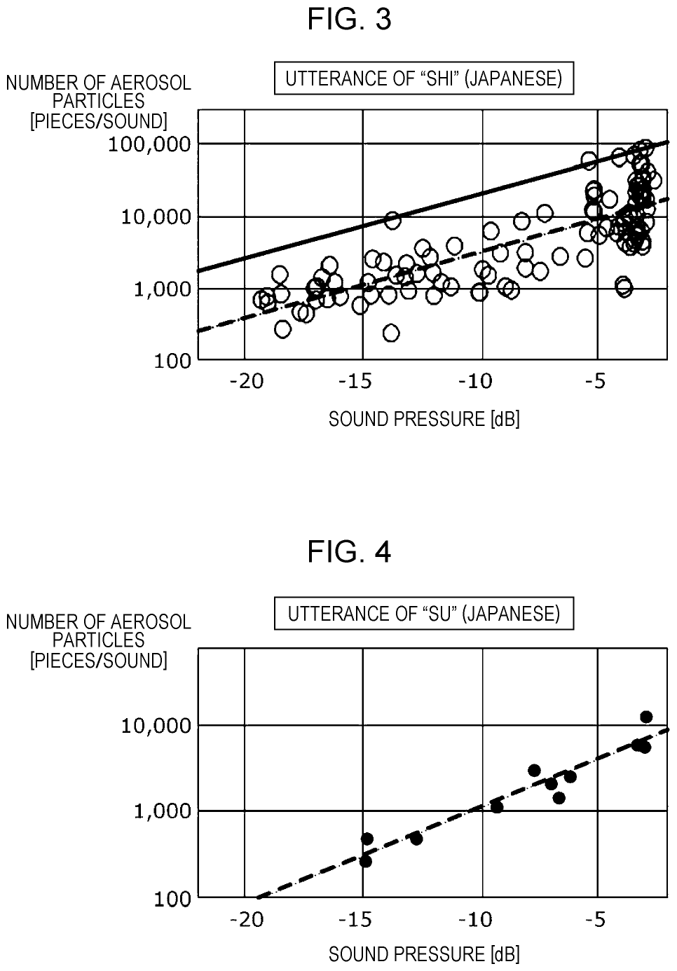

FIG. 3 is a diagram illustrating a result of measurement of sound pressure dependence of the number of aerosol particles released when a monosyllable “shi” (Japanese) is uttered once. In FIG. 3, a horizontal axis represents sound pressure [dB], and a vertical axis represents, as logarithms, the number of aerosol particles released as a result of one utterance of the monosyllable. As illustrated in FIG. 3, the number of aerosol particles released when the monosyllable “shi” (Japanese) is uttered once is up to 50,000 in the worst case (solid line). It can be seen that this is relatively large compared to other monosyllable and that about the same number of aerosol particles as by a single cough are released.

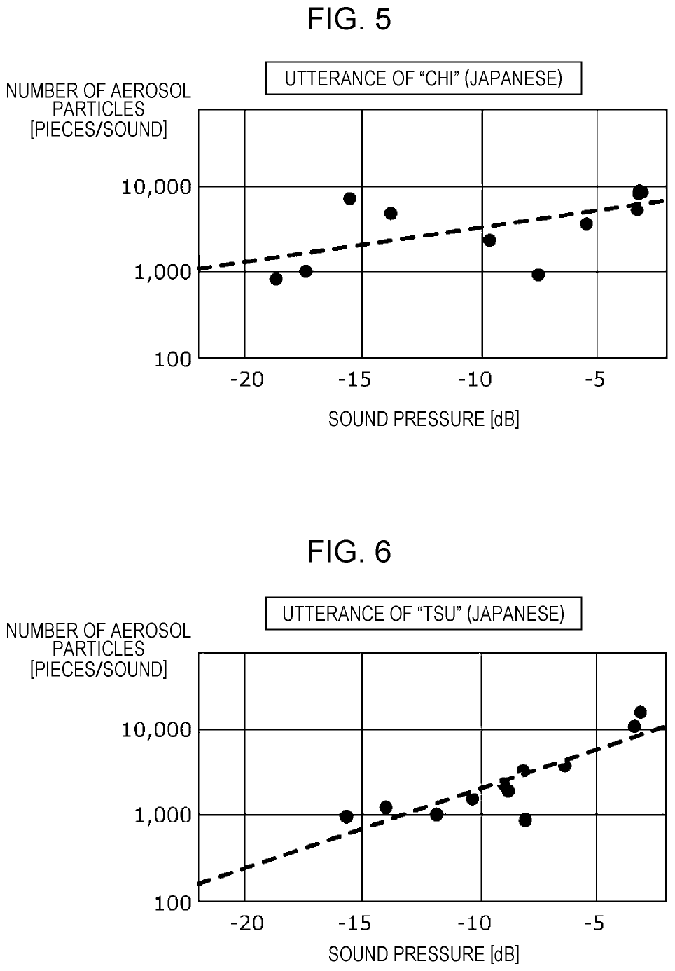

FIG. 4 is a diagram illustrating a result of measurement of sound pressure dependence of the number of aerosol particles released when a monosyllable “su” (Japanese) is uttered once. FIG. 5 is a diagram illustrating a result of measurement of sound pressure dependence of the number of aerosol particles released when a monosyllable “chi” (Japanese) is uttered once. FIG. 6 is a diagram illustrating a result of measurement of sound pressure dependence of the number of aerosol particles released when a monosyllable “tsu” (Japanese) is uttered once. In FIGS. 4, 5, and 6, horizontal axes represent sound pressure [dB], and vertical axes represent, as logarithms, the number of aerosol particles released by one utterance of the corresponding monosyllable. It can be seen from FIGS. 4, 5, and 6 that the number of aerosol particles released when each of the monosyllables “su” (Japanese), “chi” (Japanese), and “tsu” (Japanese) is uttered once is relatively large and increases up to 10,000 if the sound pressure is high.

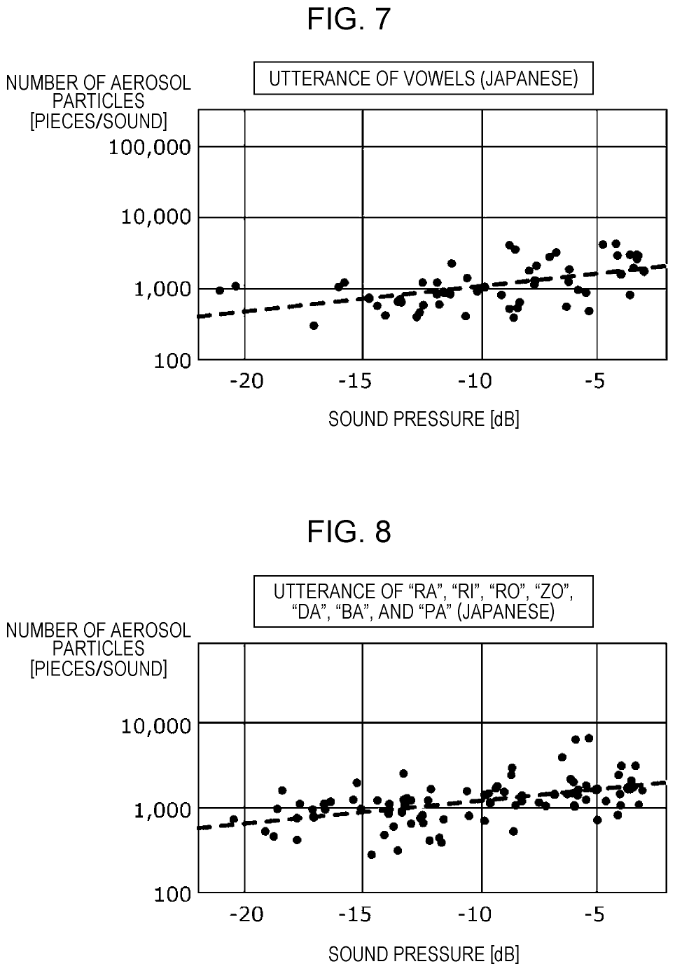

FIG. 7 is a diagram illustrating a result of measurement of sound pressure dependence of the number of aerosol particles released when monosyllables of vowels are uttered once. FIG. 8 is a diagram illustrating a result of measurement of sound pressure dependence of the number of aerosol particles released when monosyllables “ra” (Japanese), “ri” (Japanese), “ro” (Japanese), “zo” (Japanese), “da” (Japanese), “ba” (Japanese), and “pa” (Japanese) are uttered once. In FIGS. 7 and 8, horizontal axes represent sound pressure [dB], and vertical axes represent, as logarithms, the number of aerosol particles released by one utterance of the corresponding monosyllable. It can be seen from FIGS. 7 and 8 that the number of aerosol particles released when monosyllables of the vowels (i.e., the monosyllables “a” (Japanese), “i” (Japanese), “u” (Japanese), “e” (Japanese), and “o” (Japanese)) or the monosyllables “ra” (Japanese), “ri” (Japanese), “ro” (Japanese), “zo” (Japanese), “da” (Japanese), “ba” (Japanese), and “pa” (Japanese) are uttered once is relatively small.

Although Japanese Unexamined Patent Application Publication No. 2011-174624 and Japanese Unexamined Patent Application Publication No. 2020-030010 take into consideration a risk of transmission of infectious disease at a time when coughing or sneezing occurs, a large number of aerosol particles caused when an utterer utters speech sounds are not taken into consideration as described with reference to FIGS. 2 to 8.

S. Asadi et al., “Aerosol emission and superemission during human speech increase with voice loudness”, Nature Scientific Reports, February 2019, Vol. 9, No. 1 and Philip Anfinrud et al., “Visualizing Speech-Generated Oral Fluid Droplets with Laser Light Scattering”, N Engl J Med 2020; 382:2061-2063 do not quantitively describe that the number of droplets caused by a speech sound and the number of droplets caused by a cough can be close to each other or that, as in the case of a cough, a large number of aerosol particles can be caused when an utterer utters a speech sound.

The above examples of the related art thus have a problem that it is difficult to accurately estimate the number of aerosol particles caused when an utterer utters speech sounds. It is therefore undesirably difficult to reduce the risk of infection.

In order to solve the above problem, an aerosol quantity estimation system according to an aspect of the present disclosure includes a detector that detects a voice and a controller that estimates a number of aerosol particles released to a space where an utterer who has emitted the voice detected by the detector exists from a certain speech sound included in the voice on a basis of a correlation between the certain speech sound and a number of aerosol particles released from the utterer when the utterer utters the certain speech sound.

As a result, the number of aerosol particles released from an utterer to a space where the utterer exists when the utterer has uttered speech sounds can be accurately estimated.

The certain speech sound may include an utterance that uses a certain one of places of articulation used to utter speech sounds.

As a result, the number of aerosol particles released from the utterer to the space where the utterer exists when the utterer has made an utterance that uses the certain place of articulation can be accurately estimated.

The certain place of articulation may include both lips, an alveolar ridge, or a back of the alveolar ridge.

As a result, the number of aerosol particles released from the utterer to the space where the utterer exists when the utterer has made an utterance that uses at least both lips, the alveolar ridge, or the back of the alveolar ridge can be accurately estimated.

The certain speech sound may include an utterance that uses a certain one of manners of articulation used to utter speech sounds.

As a result, the number of aerosol particles released from the utterer to the space where the utterer exists when the utterer has made an utterance that uses the certain manner of articulation can be accurately estimated.

The certain manner of articulation may include a plosive, a fricative, or an affricate.

As a result, the number of aerosol particles released from the utterer to the space where the utterer exists when the utterer has made an utterance that uses at least a plosive, a fricative, or an affricate can be accurately estimated.

The certain speech sound may be an utterance “shi” in Japanese.

As a result, the number of aerosol particles released from the utterer to the space where the utterer exists when the utterer has made the utterance “shi” in Japanese, which causes a larger number of aerosol particles than other utterances, can be accurately estimated.

The correlation may include a correlation between a level of sound pressure of the certain speech sound and the number of aerosol particles released from the utterer when the utterer has uttered the certain speech sound. The controller may estimate, on a basis of the correlation, the number of aerosol particles from a detected level of sound pressure, which is detected from the certain speech sound included in the voice detected by the detector.

As a result, the number of aerosol particles corresponding to the level of sound pressure of the certain speech sound can be accurately estimated.

The aerosol quantity estimation system may further include a distance calculator that calculates a distance from the detector to the utterer who has emitted the voice. The controller may (i) calculate, on a basis of the detected level of sound pressure and the distance measured by the distance calculator, the level of sound pressure of the certain speech sound at a time when the utterer has uttered the certain speech sound and (ii) estimate the number of aerosol particles from the calculated level of sound pressure of the certain speech sound on the basis of the correlation.

In general, a level of sound pressure detected by the detector becomes lower as a distance between the detector and a sound source increases. The number of aerosol particles released from the utterer, therefore, can be estimated more accurately by calculating the level of sound pressure of the certain speech sound in accordance with the distance between the detector and the utterer.

The detector may include microphones that detect voices. The distance calculator may calculate the distance on a basis of the voices detected by the microphones.

As a result, the distance between the detector and the utterer can be calculated by detecting voices.

The aerosol quantity estimation system may further include an imager that captures an image of a voice detection range of the detector. The distance calculator may calculate the distance using the image captured by the imager.

As a result, the distance between the detector and the utterer can be calculated by capturing an image of the voice detection range of the detector.

The aerosol quantity estimation system may further include a storage storing the correlation and a communicator communicably connected to a communication network. The controller may update the correlation stored in the storage to a new correlation between the certain speech sound and the number of aerosol particles, the new correlation being obtained by the communicator through communication with an external apparatus.

As a result, the correlation stored in the storage can be updated to the new correlation. If the new correlation is more accurate, for example, the number of aerosol particles released from the utterer can be estimated more accurately by updating the correlation.

If the estimated number of aerosol particles exceeds a certain number of aerosol particles, the controller may issue a warning.

As a result, if the number of aerosol particles with which it can be determined that a risk of infection is high is exceeded, for example, a warning indicating that the risk of infection is high can be issued. The warning, therefore, can be issued at appropriate times.

The controller may issue the warning using an apparatus outside the aerosol quantity estimation system.

As a result, the warning can be issued to the utterer or a user in the space at appropriate times.

The apparatus outside the aerosol quantity estimation system may be a display apparatus provided in the space or a mobile terminal owned by the utterer.

As a result, the warning can be issued to the utterer or the user in the space at appropriate times.

The aerosol quantity estimation system may further include a body temperature measuring unit that measures body temperature of the utterer. If the body temperature measured by the body temperature measurer exceeds a certain body temperature, the controller may issue the warning.

As a result, the warning can be issued if it can be determined that the risk of infection is even higher.

If the estimated number of aerosol particles exceeds the certain number of aerosol particles, the controller may spray a disinfectant solution or radiate ultraviolet light in the space using an apparatus outside the aerosol quantity estimation system.

As a result, if the number of aerosol particles with which it can be determined that the risk of infection is high is exceeded, for example, a disinfectant solution can be sprayed or ultraviolet light can be radiated in the space. Since the disinfectant solution can be sprayed or ultraviolet light can be radiated at appropriate times, therefore, the risk of infection can be reduced.

If the estimated number of aerosol particles exceeds the certain number of aerosol particles, the controller may ventilate the space.

As a result, if the number of aerosol particles with which it can be determined that the risk of infection is high is exceeded, for example, the space can be ventilated. Since the space can effectively ventilated at appropriate times, therefore, the risk of infection can be reduced.

An aerosol quantity estimation method according to another aspect of the present disclosure includes detecting a voice and estimating a number of aerosol particles released to a space where an utterer who has emitted the detected voice exists from a certain speech sound included in the voice on a basis of a correlation between the certain speech sound and a number of aerosol particles released from the utterer when the utterer utters the certain speech sound.

As a result, the number of aerosol particles released from the utterer to the space where the utterer exists when the utterer has uttered speech sounds can be accurately estimated.

It should be noted that these general or specific aspects may be implemented as an apparatus, an integrated circuit, a computer program, a computer-readable storage medium such as a CD-ROM, or any selective combination thereof.

Embodiments will be specifically described hereinafter with reference to the drawings.

The embodiments described hereinafter are general or specific examples. Values, shapes, materials, components, arrangement positions and connection modes of the components, steps, order of the steps are examples, and are not intended to limit the present disclosure. Among the components mentioned in the following embodiments, ones not described in the independent claims will be described as optional components.

The drawings are schematic diagrams, and not necessarily strict illustrations. Scales and the like, therefore, do not necessarily match between the drawings. In the drawings, essentially the same components are given the same reference numerals, and redundant description thereof is omitted or simplified.

Ranges of values herein are not strict, and include essentially the same ranges with a difference of, say, several percent.

### First Embodiment

### 1-1. Configuration

First, the configuration of an aerosol quantity estimation system according to a first embodiment will be described.

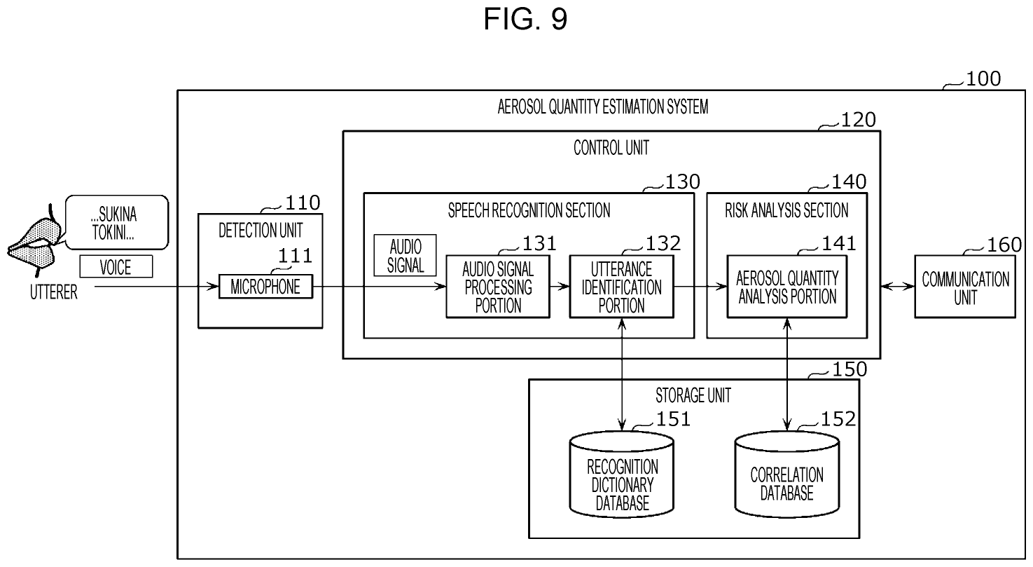

FIG. 9 is a block diagram illustrating an example of the configuration of the aerosol quantity estimation system according to the first embodiment.

An aerosol quantity estimation system 100 includes a detection unit 110, a control unit 120, a storage unit 150, and a communication unit 160.

The detection unit 110 includes a microphone 111 that detects a voice. The detection unit 110 detects, for example, a voice emitted by an utterer. The microphone 111 detects a voice emitted by an utterer and converts the detected voice into an audio signal. The audio signal obtained as a result of the conversion is output to the control unit 120.

The control unit 120 estimates, from certain speech sounds included in a voice detected by the detection unit 110 on the basis of correlations (described later) stored in the storage unit 150 in advance, the number of aerosol particles released to a space where the utterer who has emitted the voice exists. The control unit 120 includes a speech recognition section 130 and a risk analysis section 140.

The speech recognition section 130 identifies, on the basis of an audio signal, each of monosyllables included in a voice indicated by the audio signal. The speech recognition section 130 determines a level of sound pressure of each of the monosyllables on the basis of the audio signal. The speech recognition section 130 includes an audio signal processing portion 131 and an utterance identification portion 132.

The audio signal processing portion 131 performs a sound pressure determination, noise reduction, and signal processing (sound analysis) such as a spectrum transformation on an audio signal. The audio signal processing portion 131 calculates mel-frequency cepstral coefficients (MFCCs), which are feature values of speech sounds, from an audio signal as acoustic feature values. The MFCCs are feature values indicating vocal tract characteristics of an utterer and generally used in speech recognition. More specifically, the MFCCs are acoustic feature values obtained by analyzing frequency spectra of speech sounds on the basis of auditory characteristics of humans. The audio signal processing portion 131 may calculate an audio signal subjected to a mel-filterbank or a spectrogram of an audio signal as acoustic feature values. The audio signal processing portion 131 receives an audio signal, performs the above-described processing, and outputs acoustic feature values.

The utterance identification portion 132 performs, using a recognition dictionary database 151 stored in the storage unit 150, utterance identification on an audio signal (acoustic feature values) subjected to the signal processing. The utterance identification portion 132 performs utterance identification based on machine learning. That is, the utterance identification portion 132 identifies each of monosyllables included in speech sounds included in a voice indicated by an audio signal. A result of the utterance identification is output to the risk analysis section 140. The utterance identification portion 132 determines intensity of each of the identified monosyllables.

The utterance identification portion 132 receives acoustic feature values, performs the above-described processing, and outputs information indicating each of one or more identified monosyllables. The utterance identification portion 132 may output the information indicating each of the one or more monosyllables and a level of sound pressure corresponding to the monosyllable while associating the information and the level of sound pressure with each other.

The recognition dictionary database 151 is, for example, a dictionary for recognizing monosyllables in speech sounds and includes a machine learning model obtained through machine learning based on a large number of training data sets, which are pairs of acoustic feature values and correct data regarding a monosyllable in a speech sound.

The recognition dictionary database 151 need not necessarily include a dictionary for recognizing all monosyllables in speech sounds, and may include only a dictionary for recognizing one or more monosyllables in certain speech sounds. In this case, the utterance identification portion 132 may identify monosyllables uttered by an utterer only for certain speech sounds.

The recognition dictionary database 151 may include a dictionary for recognizing one or more monosyllables in certain speech sounds (one or more first monosyllables) and need not include a dictionary for recognizing monosyllables (one or more second monosyllables) other than the one or more first monosyllables. In this case, the utterance identification portion 132 may identify each of the one or more first monosyllables uttered by an utterer and need not identify each of the one or more second monosyllables uttered by the utterer.

The risk analysis section 140 estimates the number of aerosol particles for each monosyllable from a result of utterance identification obtained from the speech recognition section 130. The risk analysis section 140 includes an aerosol quantity analysis portion 141.

The aerosol quantity analysis portion 141 estimates the number of aerosol particles for each monosyllable included in a result of utterance identification using a correlation database 152 stored in the storage unit 150. The aerosol quantity analysis portion 141 estimates, on the basis of correlations, the number of aerosol particles from levels of sound pressure detected from certain speech sounds included in a voice detected by the detection unit 110. For example, the aerosol quantity analysis portion 141 estimates the number of aerosol particles caused when an utterer has uttered each monosyllable included in a result of utterance identification performed by the utterance identification portion 132 by reading a correlation corresponding to the monosyllable from the correlation database 152 and identifying the number of aerosol particles corresponding, in the read correlation, to a level of sound pressure of the monosyllable. The aerosol quantity analysis portion 141 adds up the number of aerosol particles for monosyllables and sequentially calculates the total number of aerosol particles caused when speech sounds included in an audio signal have been uttered. The aerosol quantity analysis portion 141 may estimate a risk of viral infection on the basis of an obtained total value.

The aerosol quantity analysis portion 141 receives information indicating one or more monosyllables and one or more levels of sound pressure corresponding to the one or more monosyllables, refers to the correlation database 152, and determines one or more values of the number of aerosol particles. The aerosol quantity analysis portion 141 determines the number of aerosol particles corresponding to an audio signal on the basis of the determined one or more values of the number of aerosol particles.

The number of aerosol particles corresponding the audio signal may be a total value obtained by adding up the determined one or more values of the number of aerosol particles.

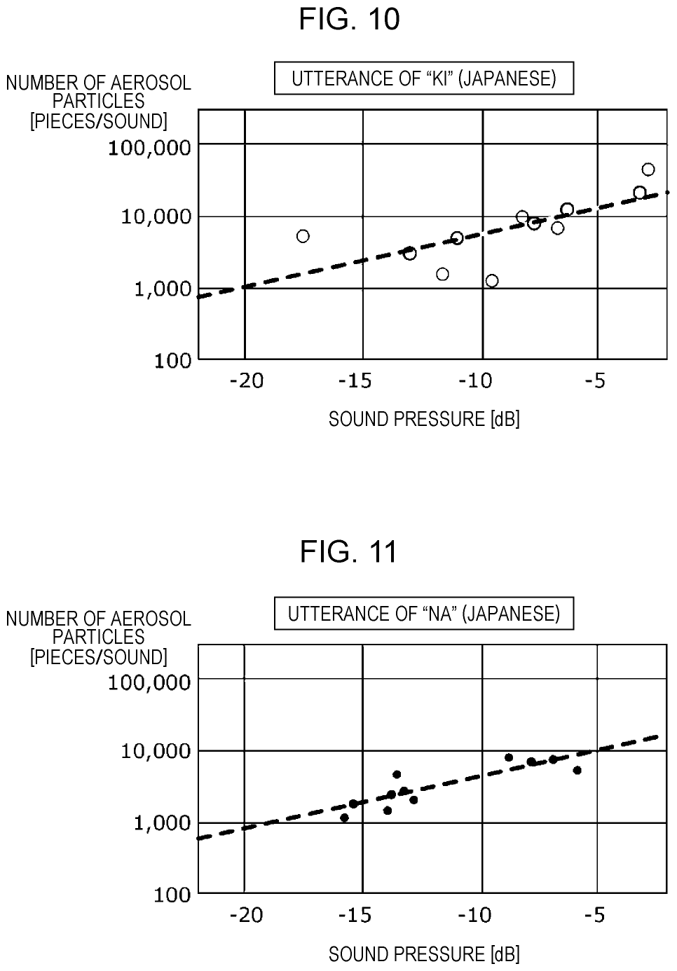

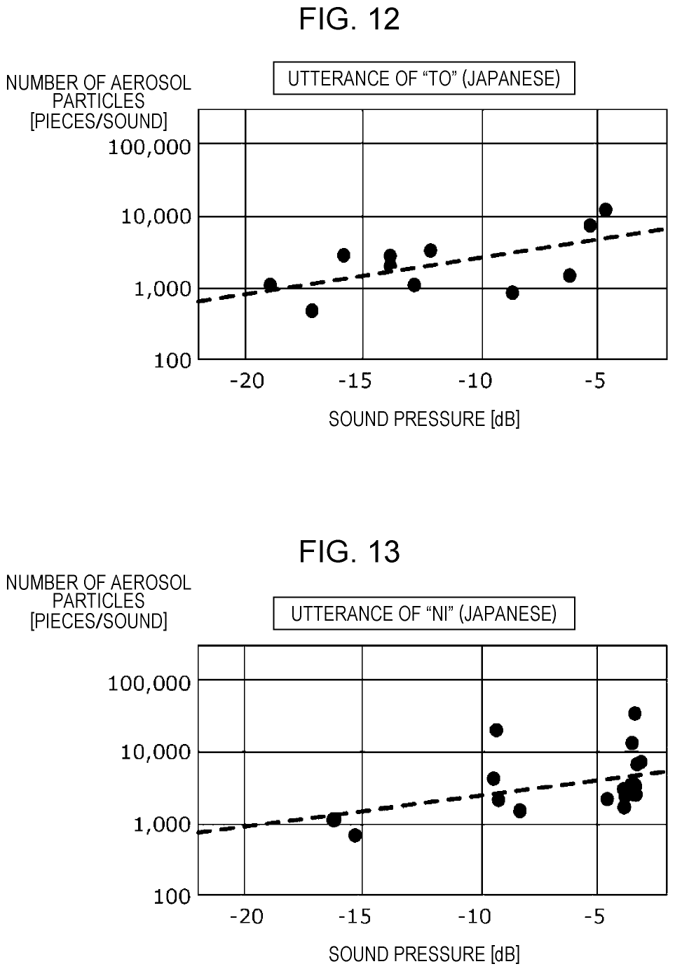

The correlation database 152 includes correlations indicating relationships between certain speech sounds and the number of aerosol particles released from an utterer when the utterer utters the certain speech sounds. The correlations indicate, as in FIGS. 2 to 8 and 10 to 13, for example, sound pressure dependence of the number of aerosol particles released when monosyllables in speech sounds are uttered. The correlations included in the correlation database 152 may be information indicating the number of aerosol particles for different monosyllables in relation to the sound pressure. The information indicating the number of aerosol particles for different monosyllables in relation to the sound pressure may be equations indicated by broken lines or solid lines in FIGS. 3 to 8 and 10 to 13. The equations indicated by the broken lines or the solid lines are regression line equations and can be obtained, for example, through a least squares method. FIGS. 10 to 13 are diagrams illustrating results of measurement of sound pressure dependence of the number of aerosol particles released when monosyllables “ki” (Japanese), “na” (Japanese), “to” (Japanese), and “ni” (Japanese) are uttered once, respectively. The correlation database 152 thus includes correlations between levels of sound pressure at a time when an utterer utters a monosyllable and the number of aerosol particles released from the utterer.

The correlation database 152 need not necessarily include correlations regarding all monosyllables in speech sounds, and may include only correlations regarding one or more monosyllables in certain speech sounds. In this case, the aerosol quantity analysis portion 141 may estimate the number of aerosol particles caused by monosyllables uttered by an utterer only for the certain speech sounds.

The correlation database 152 may include correlations regarding one or more monosyllables in certain speech sounds (one or more first monosyllables) and need not include correlations regarding monosyllables other than the one or more first monosyllables (one or more second monosyllables). In this case, the aerosol quantity analysis portion 141 may estimate the number of aerosol particles caused by each of the one or more first monosyllables uttered by an utterer and need not estimate the number of aerosol particles caused by each of the one or more second monosyllables uttered by the utterer.

The certain speech sounds may include utterances that use certain ones of places of articulation used to utter speech sounds. The certain places of articulation include, for example, both lips, the alveolar ridge, and the back of the alveolar ridge.

The certain speech sounds may include utterances that use certain ones of manners of articulation used to utter speech sounds. The certain manners of articulation include, for example, a plosive, a fricative, and an affricate.

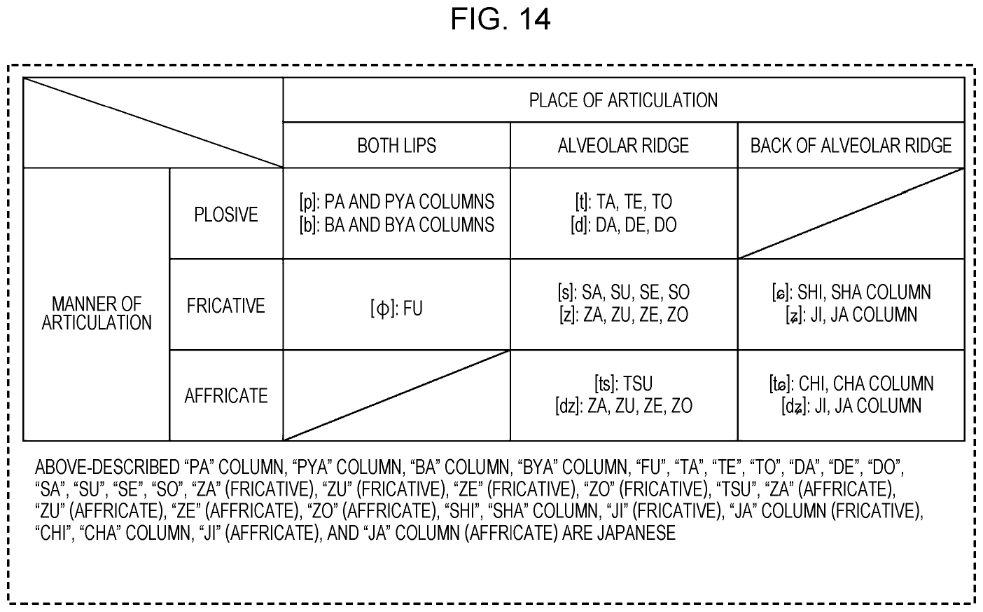

As illustrated in FIG. 14, for example, the certain speech sounds may be utterances that use one of the places of articulation including both lips, the alveolar ridge, and the back of the alveolar ridge and one of the manners of articulation including a plosive, a fricative, and an affricate. FIG. 14 is a table illustrating an example of the certain speech sounds. As illustrated in FIG. 14, the certain speech sounds may include utterances in languages other than Japanese, in addition to utterances in Japanese. That is, the certain speech sounds are not limited to utterances in Japanese and also include utterances in languages other than Japanese, such as English, Chinese, and French, that is, for example, utterances indicated by the International Phonetic Alphabet (IPA). The IPA is a phonetic representation of a speech sound in any language based on a way the speech sound is uttered, and an utterance “shi” (Japanese), for example, is represented as follows.
[θ][i], [c][i], [f][i], and [s][i]

One speech sound might thus correspond to different representations of the IPA. Utterance identification is herein defined as identification of phonetic symbols, monosyllables, pronunciations, and words.

The certain speech sounds include, for example, at least one of utterances represented by [p] (“pa” column (Japanese) and “pya” column (Japanese)), utterances represented by [b] (“ba” column (Japanese) and “bya” column (Japanese)), an utterance represented by [ϕ] (“fu” (Japanese)), utterances represented by [t] (“ta” (Japanese), “te” (Japanese), and “to” (Japanese)), utterances represented by [d] (“da” (Japanese), “de” (Japanese), and “do” (Japanese)), utterances represented by [s] (“sa” (Japanese), “su” (Japanese), “se” (Japanese), and “so” (Japanese)), utterances represented by [z] (“za” (Japanese), “zu” (Japanese), “ze” (Japanese), and “zo” (Japanese)), an utterance represented by [ts] (“tsu” (Japanese)), and utterances represented by [dz] (“za” (Japanese), “zu” (Japanese), “ze” (Japanese), and “zo” (Japanese)). The certain speech sounds also include at least one of utterances represented by [ [symbol] ] (“shi” (Japanese) and “sha” column (Japanese)), utterances represented by [ [symbol] ] Cji” (Japanese) and “ja” column (Japanese)), utterances represented by [t [symbol] ] (“chi” (Japanese) and “cha” column (Japanese)), and utterances represented by [d [symbol] ] (“ji” (Japanese) and “ja” column (Japanese)).

The aerosol quantity analysis portion 141 may determine whether a calculated total value exceeds a certain number of aerosol particles and, if the total value exceeds the certain number of aerosol particles, determine that a risk of transmission of infectious disease is high. The certain number of aerosol particles is stored, for example, in the storage unit 150. The aerosol quantity analysis portion 141 may issue a warning if the calculated total value exceeds the certain number of aerosol particles. For example, the aerosol quantity analysis portion 141 may issue a warning by controlling the communication unit 160 such that the communication unit 160 transmits warning information indicating the warning to an external apparatus outside the aerosol quantity estimation system 100. The external apparatus to which the warning information is transmitted may be, for example, a display apparatus provided in a space where the microphone 111 of the aerosol quantity estimation system 100 is provided or a mobile terminal owned by the utterer. An address indicating the display apparatus or the mobile terminal in this case may be registered to the aerosol quantity estimation system 100 in advance. The display apparatus may be included in the aerosol quantity estimation system 100 and display a result of processing performed by the aerosol quantity analysis portion 141.

If determining that the risk of infection is high, the aerosol quantity analysis portion 141 may notify the utterer of the result of the determination (e.g., a warning indicating a high risk of infection), and if determining that the risk of infection is not high, the aerosol quantity analysis portion 141 need not notify the utterer of the result of the determination. Since a warning is issued if the risk of infection is high, the user can be prompted to take measures to reduce the number of aerosol particles.

The aerosol quantity analysis portion 141 may determine the risk of infection itself, instead of determining whether the risk of infection is high. For example, the aerosol quantity analysis portion 141 may determine a higher risk of infection as the total value increases. The result of the determination may be classifications of different levels such as “there is some risk of infection”, “risk of infection is high”, and “risk of infection is very high”, or may be a value indicating the risk of infection.

The storage unit 150 stores the recognition dictionary database 151 and the correlation database 152.

The communication unit 160 is communicably connected to a communication network. The communication unit 160 may communicate with an external apparatus (e.g., a server) outside the aerosol quantity estimation system 100 over the communication network. The communication unit 160 may communicate with the server, for example, and receive new correlations between certain speech sounds and the number of aerosol particles from the server. The new correlations may be, for example, more accurate correlations generated by the server using more results of experiments or the like. When the communication unit 160 receives new correlations, the control unit 120 controls the communication unit 160 such that the correlations stored in the correlation database 152 of the storage unit 150 are updated to the new correlations received by the communication unit 160. The communication unit 160 need not necessarily receive new correlations from the server, and may receive a new dictionary for improving accuracy of identifying utterances or a certain new number of aerosol particles (threshold) for issuing a warning. In this case, when the communication unit 160 receives the new dictionary from the server, the control unit 120 may control the communication unit 160 such that the dictionary stored in the recognition dictionary database 151 of the storage unit 150 is updated to the new dictionary received by the communication unit 160. When the communication unit 160 receives the new certain number of aerosol particles (threshold) from the server, the control unit 120 may control the communication unit 160 such that the certain number of aerosol particles (threshold) stored in the storage unit 150 is updated to the certain new number of aerosol particles (threshold).

The communication unit 160 may obtain infection information indicating a relationship between the number of aerosol particles and a risk of viral infection or a relationship between the number of aerosol particles and a state of infection from the server. The infection information may be used in the determination of the risk of infection made by the aerosol quantity analysis portion 141.

### 1-2. Operation

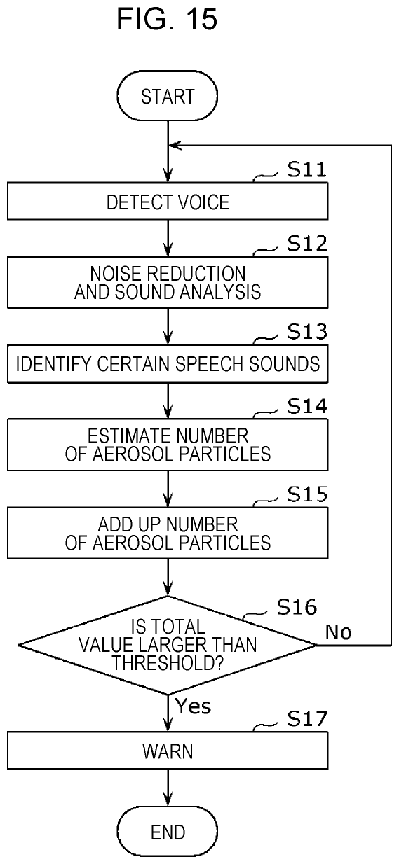

Next, an operation performed by the aerosol quantity estimation system 100 according to the present embodiment will be described with reference to FIG. 15. FIG. 15 is a flowchart illustrating an example of the operation performed by the aerosol quantity estimation system according to the present embodiment.

First, in the aerosol quantity estimation system 100, the detection unit 110 detects a voice with the microphone 111 and generates an audio signal (S11).

Next, the audio signal processing portion 131 of the speech recognition section 130 in the control unit 120 reduces noise in the generated audio signal and performs signal processing (sound analysis) such as a spectrum transformation (S12).

Next, the utterance identification portion 132 of the speech recognition section 130 performs, using the recognition dictionary database 151, utterance identification on the audio signal subjected to the signal processing. As a result, the utterance identification portion 132 identifies certain speech sounds, namely “shi” (Japanese), “su” (Japanese), “chi” (Japanese), “tsu” (Japanese), “ki” (Japanese), and “to” (Japanese), for example, among monosyllables included in speech sounds included in the voice indicated by the audio signal (S13). The utterance identification portion 132 may identify all speech sounds including the certain speech sounds among the monosyllables included in the speech sounds included in the voice indicated by the audio signal.

Next, the aerosol quantity analysis portion 141 of the risk analysis section 140 estimates the number of aerosol particles for each monosyllable included in a result of the utterance identification using the correlation database 152 (S14). The aerosol quantity analysis portion 141 estimates the number of aerosol particles caused when an utterer has uttered each monosyllable included in the result of the utterance identification performed by the utterance identification portion 132 by reading a correlation corresponding to the monosyllable from the correlation database 152 and identifying the number of aerosol particles corresponding, in the read correlation, to a level of sound pressure of the monosyllable.

Next, the aerosol quantity analysis portion 141 adds up the number of aerosol particles for each monosyllable estimated in step S14 (S15). The aerosol quantity analysis portion 141 stores a total value in the storage unit 150. As a result, the aerosol quantity analysis portion 141 can calculate the total value (cumulative value) by adding the number of aerosol particles estimated in step S14 to a total value obtained in a previous addition. If the previous total value is not stored in the storage unit 150, the total value is calculated with the previous total value assumed as 0.

Next, the aerosol quantity analysis portion 141 determines whether the total value of the number of aerosol particles is larger than a certain number of aerosol particles (S16).

If the total value of the number of aerosol particles is larger than the certain number of aerosol particles (YES in S16), the aerosol quantity analysis portion 141 issues a warning (S17). If the total value of the number of aerosol particles is smaller than or equal to the certain number of aerosol particles (NO in S16), the aerosol quantity analysis portion 141 returns to step S11. That is, after calculating the total value of the number of aerosol particles or if determining that a warning need not be issued, the aerosol quantity analysis portion 141 performs the same process again. The aerosol quantity estimation system 100 may keep performing the same process even after a warning is issued.

### 1-3. Operation

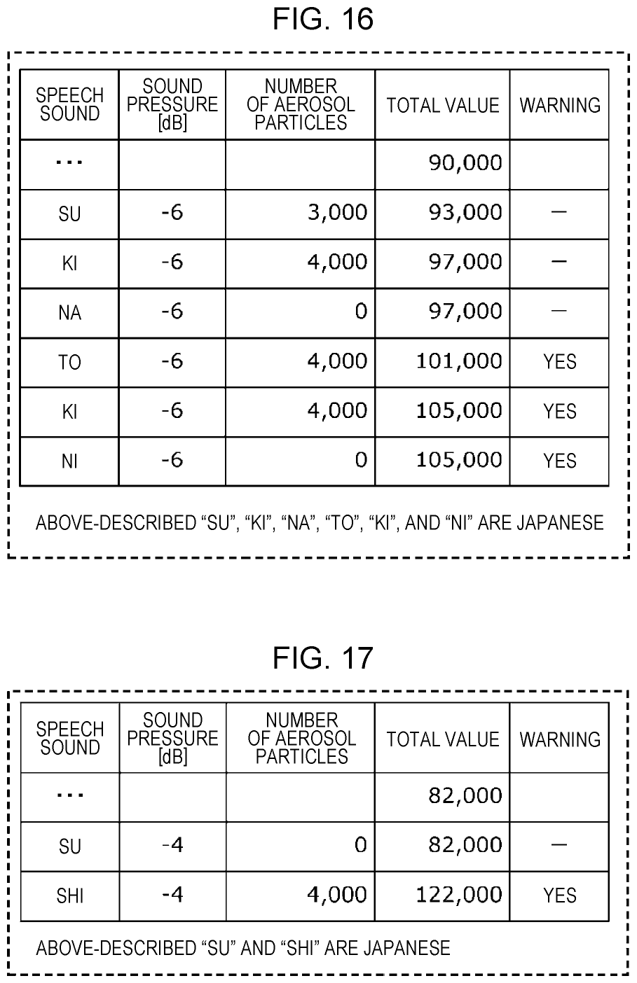

Next, a specific example of the process performed by the aerosol quantity analysis portion 141 of the risk analysis section 140 will be described with reference to FIG. 16. FIG. 16 is a diagram illustrating a specific example of the process performed by the aerosol quantity analysis portion. FIG. 16 is specifically a table indicating levels of sound pressure of monosyllables in speech sounds, the estimated number of aerosol particles, total values, and presence or absence of a warning.

The aerosol quantity analysis portion 141 calculates the number of aerosol particles for, among monosyllables “su” (Japanese), “ki” (Japanese), “na” (Japanese), “to” (Japanese), “ki”, and “ni” (Japanese) in speech sounds identified by the utterance identification portion 132, certain speech sounds, namely “su” (Japanese), “ki” (Japanese), “to” (Japanese), and “ki” (Japanese), for example, by identifying the number of aerosol particles corresponding, in corresponding correlations, to levels of sound pressure of the certain speech sounds. The aerosol quantity analysis portion 141 then adds up the calculated number of aerosol particles and, if a total value exceeds the certain number of aerosol particles (threshold), namely 100,000, for example, issues a warning. In this case, two different thresholds, namely 50,000 and 100,000, for example, may be set in order to issue a warning, and the aerosol quantity analysis portion 141 may issue a caution when the total value exceeds 50,000 and then issue a warning when the total value exceeds 100,000. The number of notifications is not limited to two, and three or more notifications may be issued, instead. After the notifications are issued, the total value stored in the storage unit 150 may be reset to 0.

### 1-4. Advantageous Effects

As described above, the aerosol quantity estimation system 100 according to the present embodiment includes the detection unit 110 that detects a voice and the control unit 120. The control unit 120 estimates the number of aerosol particles released to a space where an utterer who has uttered speech sounds exists from certain speech sounds included in the voice detected by the detection unit 110 on the basis of correlations between the certain speech sounds and the number of aerosol particles released from the utterer when the utterer utters the certain speech sounds.

The number of aerosol particles released from an utterer to a space where the utterer exists when the utterer has uttered speech sounds, therefore, can be accurately estimated.

In the aerosol quantity estimation system 100, the certain speech sounds include utterances that use certain ones of places of articulation used to utter speech sounds. The number of aerosol particles released from an utterer to a space where the utterer exists when the utterer has made utterances using the certain places of articulation, therefore, can be accurately estimated.

In the aerosol quantity estimation system 100, the certain places of articulation include both lips, the alveolar ridge, and the back of the alveolar ridge. The number of aerosol particles released from an utterer to a space where the utterer exists when the utterer has made an utterance that uses at least both lips, the alveolar ridge, or the back of the alveolar ridge can be accurately estimated.

In the aerosol quantity estimation system 100, the certain speech sounds include utterances that use certain ones of manners of articulation used to utter speech sounds. The number of aerosol particles released from an utterer to a space where the utterer exists when the utterer has made utterances using the certain manners of articulation, therefore, can be accurately estimated.

In the aerosol quantity estimation system 100, the certain manners of articulation include a plosive, a fricative, and an affricate. The number of aerosol particles released from an utterer to a space where the utterer exists when the utterer has made an utterance using at least a plosive, a fricative, or an affricate, therefore, can be accurately estimated.

In the aerosol quantity estimation system 100, the correlations include correlations between levels of sound pressure of the certain speech sounds at a time when an utterer utters the certain speech sounds and the number of aerosol particles released from the utterer. The control unit 120 estimates, on the basis of the correlations, the number of aerosol particles from levels of sound pressure detected from the certain speech sounds included in a voice detected by the detection unit 110. The number of aerosol particles corresponding to the levels of sound pressure of the certain speech sounds, therefore, can be accurately estimated.

The aerosol quantity estimation system 100 also includes the storage unit 150 storing the correlations and the communication unit 160 communicably connected to the communication network. The control unit 120 updates the correlations stored in the storage unit 150 to new correlations between the certain speech sounds and the number of aerosol particles obtained as a result of communication between the communication unit 160 and an external apparatus.

The correlations stored in the storage unit 150, therefore, can be updated to the new correlations. If the new correlations are more accurate, for example, the number of aerosol particles released from an utterer can be estimated more accurately by updating the correlations.

### 1-5. Modification

Next, a modification of the first embodiment will be described.

In an aerosol quantity estimation system 100 according to the modification of the first embodiment, the utterance identification portion 132 may identify only the utterance “shi” (Japanese), for example, as a certain speech sound. The aerosol quantity analysis portion 141 may then estimate the number of aerosol particles released to a space where an utterer who has emitted a voice exists from only “shi” (Japanese) included in the voice emitted by the utterer. That is, the certain speech sounds may include only the utterance “shi” (Japanese). The certain speech sounds may also include, as in the first embodiment, other utterances equivalent to the utterance in Japanese.

The utterance identification portion 132 may identify the utterance “shi” (Japanese) and need not identify utterances other than the utterance “shi” (Japanese). The aerosol quantity analysis portion 141 may estimate, from speech sounds identified as “shi” (Japanese) included in a voice emitted by an utterer, the number of aerosol particles released to a space where the utterer who has emitted the voice exists, and need not estimate, from speech sounds that are not identified as “shi” (Japanese) included in the voice emitted by the utterer, the number of aerosol particles released to the space where the utterer who has emitted the voice exists.

The utterance identification portion 132 may identify all monosyllables in speech sounds instead of identifying only the utterance “shi” (Japanese), and the aerosol quantity analysis portion 141 may estimate the number of aerosol particles released to the space when speech sounds identified as “shi” (Japanese) have been uttered.

The utterance identification portion 132 may identify all monosyllables in speech sounds instead of identifying the utterance “shi” (Japanese) and not identifying the utterances other than “shi” (Japanese). The aerosol quantity analysis portion 141 may estimate the number of aerosol particles released to the space when speech sounds identified as “shi” (Japanese) have been uttered and need not estimate the number of aerosol particles released to the space when speech sounds that are not identified as “shi” (Japanese) have been uttered.

As indicated by the solid line in FIG. 3, when the monosyllable “shi” (Japanese) is uttered, about the same number of aerosol particles as by a cough are caused in the worst case. In this case, the correlation database 152 may store only a correlation regarding the monosyllable “shi” (Japanese) illustrated in FIG. 3, and the aerosol quantity analysis portion 141 may estimate the number of aerosol particles in the worst case corresponding to only levels of sound pressure at times when the monosyllable “shi” (Japanese) has been uttered in a voice.

The correlation database 152 may store the correlation regarding the monosyllable “shi” (Japanese) illustrated in FIG. 3 and need not store correlations regarding monosyllables other than “shi” (Japanese). The aerosol quantity analysis portion 141 may estimate, using the correlation database 152, the number of aerosol particles in the worst case corresponding to levels of sound pressure at times when the monosyllable “shi” (Japanese) has been uttered in a voice, and need not estimate the number of aerosol particles corresponding to levels of sound pressure at times when monosyllables other than “shi” (Japanese) have been uttered in the voice.

Next, a specific example of a process performed by the risk analysis section 140 according to the modification of the first embodiment will be described with reference to FIG. 17. FIG. 17 is a diagram illustrating the specific example of the process performed by the risk analysis section according to the modification of the first embodiment. FIG. 17 is specifically a table indicating levels of sound pressure of monosyllables in speech sounds, the estimated number of aerosol particles, total values, and presence or absence of a warning.

The risk analysis section 140 calculates the number of aerosol particles for each monosyllable in speech sounds identified by the utterance identification portion 132 by identifying the number of aerosol particles corresponding, in a corresponding correlation, to a level of sound pressure of, among “su” (Japanese) and “shi” (Japanese), which are monosyllables in the speech sounds, for example, the certain speech sound “shi”. The risk analysis section 140 then adds the calculated number of aerosol particles and, if the certain number of aerosol particles (threshold), namely 100,000, for example, is exceeded, issues a warning. In this case, two different thresholds, namely 50,000 and 100,000, for example, may be set in order to issue a warning, and the risk analysis section 140 may issue a caution when the total value exceeds 50,000 and then issue a warning when the total value exceeds 100,000. The number of notifications is not limited to two, and three or more notifications may be issued. After the notifications are issued, the total value stored in the storage unit 150 may be reset to 0.

In the aerosol quantity estimation system 100 according to the modification of the first embodiment, the certain speech sounds include only the utterance “shi” (Japanese). The number of aerosol particles released from an utterer to a space where the utterer exists when the utterer has uttered “shi” (Japanese), which causes a large number of aerosol particles than other utterances, therefore, can be accurately estimated.

### Second Embodiment

### 2-1. Configuration

Next, the configuration of an aerosol quantity estimation system according to a second embodiment will be described.

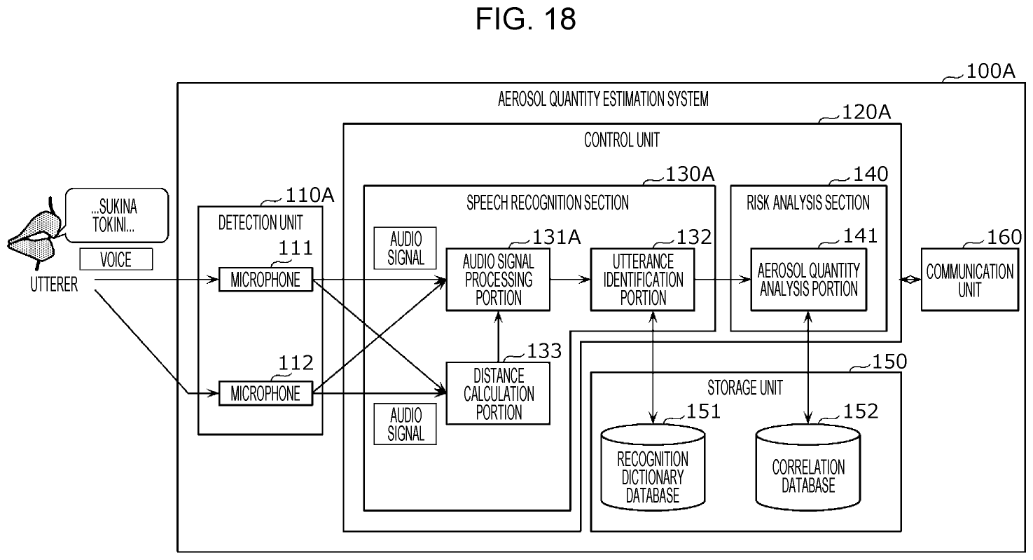

FIG. 18 is a block diagram illustrating an example of the configuration of the aerosol quantity estimation system according to the second embodiment.

An aerosol quantity estimation system 100A according to the second embodiment is different from the aerosol quantity estimation system 100 according to the first embodiment in terms of the configuration of a detection unit 110A and a control unit 120A. More specifically, the aerosol quantity estimation system 100A is different from the aerosol quantity estimation system 100 in that the detection unit 110A includes microphones 111 and 112. The aerosol quantity estimation system 100A is different from the aerosol quantity estimation system 100 in that a speech recognition section 130A included in the control unit 120A includes a distance calculation portion 133 that identifies a position at which a sound has been caused, that is, more specifically, calculates a distance from the detection unit 110A to a position (utterer) at which the sound has been caused, using results of detection performed by the microphones 111 and 112. Processing performed by an audio signal processing portion 131A is also different.

The microphones 111 and 112 are provided at different positions. The microphones 111 and 112 each detect a voice.

The distance calculation portion 133 calculates a distance from the detection unit 110A to an utterer who has emitted a voice. The distance calculation portion 133 calculates the distance on the basis of voices detected by the microphones 111 and 112. For example, the distance calculation portion 133 estimates, using audio signals generated by the microphones 111 and 112, a distance L from the detection unit 110A to the utterer through trigonometry on the basis of a difference in arrival time or phase of sound waves at the two different positions at which the microphones 111 and 112 are provided.

The audio signal processing portion 131A calculates (corrects) a level of sound pressure of a certain speech sound at a time when an utterer has uttered the certain speech sound on the basis of a level of sound pressure of a voice detected by the detection unit 110A and a distance between the detection unit 110A and the utterer calculated by the distance calculation portion 133. That is, the audio signal processing portion 131A calculates attenuation ΔP [dB] of the voice emitted by the utterer over a distance from the utterer's mouth to the detection unit 110A and corrects a level of sound pressure of the voice based on the voice detected by the detection unit 110A to calculate a level of sound pressure of the voice at the utterer's mouth. The attenuation ΔP [dB] of the voice at this time is represented by the following expression (1), where L₀ denotes a reference distance. L₀ is, for example, 1 m.

ΔPi=20×log₁₀(Li/L₀) (1)

A level of sound pressure Bi of the voice at the sound source, that is, the utterer's mouth, before the attenuation is therefore represented by the following expression (2).

Bi=Ai+ΔPi (2)

That is, the corrected level of sound pressure Bi that takes into consideration the distance, that is, the level of sound pressure of the voice emitted by the utterer at the utterer's mouth, is calculated from a measured level of sound pressure Ai.

The aerosol quantity analysis portion 141 estimates the number of aerosol particles from the corrected level of sound pressure (corrected level of sound pressure Bi) on the basis of a correlation regarding an identified monosyllable. That is, the aerosol quantity analysis portion 141 reads the correlation regarding the identified monosyllable from the correlation database 152 and identifies the number of aerosol particles corresponding, in the read correlation, to the corrected level of sound pressure of the identified monosyllable to estimate the number of aerosol particles caused when the utterer has uttered the monosyllable.

### 2-2. Operation

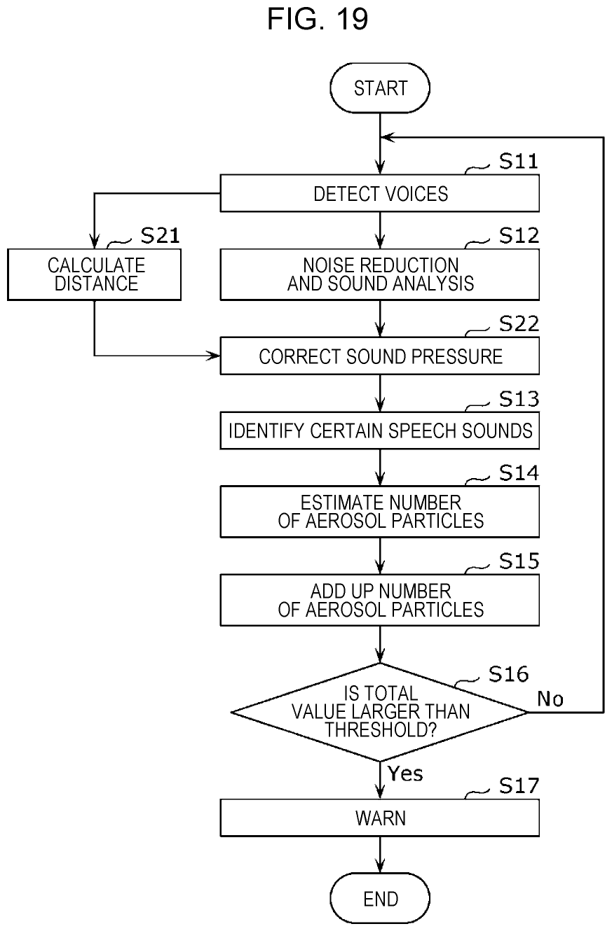

Next, an operation performed by the aerosol quantity estimation system 100A according to the present embodiment will be described with reference to FIG. 19. FIG. 19 is a flowchart illustrating an example of the operation performed by the aerosol quantity estimation system according to the present embodiment.

First, in the aerosol quantity estimation system 100A, the detection unit 110A detects voices with the microphones 111 and 112 and generates audio signals (S11).

Next, the audio signal processing portion 131A of the speech recognition section 130A in the control unit 120A reduces noise in the generated audio signals and performs signal processing (speech analysis) such as a spectrum transformation (S12).

In parallel with step S12, the distance calculation portion 133 of the speech recognition section 130A calculates a distance to an utterer who has emitted a voice on the basis of the detected audio signals (S21).

The audio signal processing portion 131A then corrects levels of sound pressure obtained on the basis of the audio signals generated by the detection unit 110A on the basis of the distance calculated by the distance calculation portion 133 (S22).

Next, the utterance identification portion 132 of the speech recognition section 130A performs, using the recognition dictionary database 151, utterance identification on the audio signals subjected to the signal processing. As a result, the utterance identification portion 132 identifies, among monosyllables included in speech sounds included in the voices indicated by the audio signals, for example, certain speech sounds, namely, for example, “shi” (Japanese), “su” (Japanese), “chi” (Japanese), “tsu” (Japanese), “ki” (Japanese), and “to” (Japanese) (S13). The utterance identification portion 132 may identify all speech sounds including the certain speech sounds among the monosyllables included in the speech sounds included in the voices indicated by the audio signals.

Next, the aerosol quantity analysis portion 141 of the risk analysis section 140 estimates, using the correlation database 152, the number of aerosol particles for each monosyllable included in a result of the utterance identification (S14). The aerosol quantity analysis portion 141 estimates the number of aerosol particles caused when the utterer has uttered each monosyllable identified by the utterance identification portion 132 by reading a correlation regarding the monosyllable from the correlation database 152 and identifying the number of aerosol particles corresponding, in the read correlation, to the corrected level of sound pressure of the monosyllable.

Next, the aerosol quantity analysis portion 141 adds up the number of aerosol particles for the monosyllables estimated in step S14 (S15). The aerosol quantity analysis portion 141 stores a total value in the storage unit 150. The aerosol quantity analysis portion 141 can thus calculate the total value (cumulative value) by adding the number of aerosol particles estimated in step S14 to a total value calculated in a previous addition. If the previous total value is not stored in the storage unit 150, the total value is calculated with the previous total value assumed as 0.

Next, the aerosol quantity analysis portion 141 determines whether the total value of the number of aerosol particles is larger than the certain number of aerosol particles (S16).

If the total value of the number of aerosol particles is larger than the certain number of aerosol particles (YES in S16), the aerosol quantity analysis portion 141 issues a warning (S17). If the total value of the number of aerosol particles is smaller than or equal to the certain number of aerosol particles (NO in S16), the aerosol quantity analysis portion 141 returns to step S11. That is, after calculating the total value of the number of aerosol particles, or if determining that a warning need not be issued, the aerosol quantity analysis portion 141 performs the same process again. The aerosol quantity estimation system 100A may keep performing the same process even after a warning is issued.

### 2-3. Operation

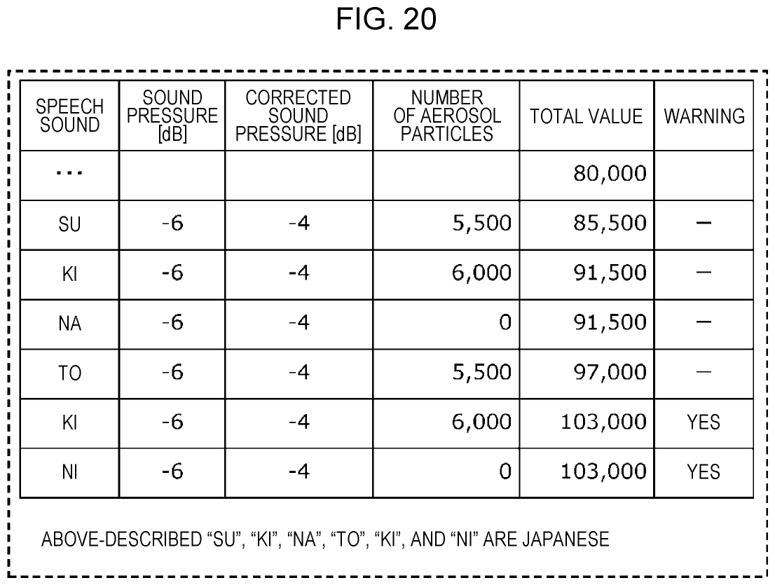

Next, a specific example of the process performed by the aerosol quantity analysis portion 141 of the risk analysis section 140 will be described with reference to FIG. 20. FIG. 20 is a diagram illustrating a specific example of the process performed by the aerosol quantity analysis portion. FIG. 20 is specifically a table indicating levels of sound pressure of monosyllables in speech sounds, the estimated number of aerosol particles, total values, and presence or absence of a warning.

The aerosol quantity analysis portion 141 calculates the number of aerosol particles for monosyllables in speech sounds identified by the utterance identification portion 132 by identifying the number of aerosol particles corresponding, in corresponding correlations, to corrected levels of sound pressure of, among “su” (Japanese), “ki” (Japanese), “na” (Japanese), “to” (Japanese), “ki”, and “ni” (Japanese), which are monosyllables in speech sounds, for example, certain speech sounds “su” (Japanese), “ki” (Japanese), “to” (Japanese), and “ki” (Japanese). The aerosol quantity analysis portion 141 then adds up the calculated number of aerosol particles and, if a total value exceeds the certain number of aerosol particles (threshold), namely 100,000, for example, issues a warning. In this case, two different thresholds, namely 50,000 and 100,000, for example, may be set in order to issue a warning, and the aerosol quantity analysis portion 141 may issue a caution when the total value exceeds 50,000 and then issue a warning when the total value exceeds 100,000. The number of notifications is not limited to two, and three or more notifications may be issued. After the notifications are issued, the total value stored in the storage unit 150 may be reset to 0.

### 2-4. Advantageous Effects

As described above, the aerosol quantity estimation system 100A according to the present embodiment also includes the distance calculation portion 133 that calculates a distance from the detection unit 110A to an utterer who has emitted a voice. The control unit 120A calculates a level of sound pressure of a certain speech sound at a time when an utterer has uttered the certain speech sound on the basis of a detected level of sound pressure and a distance measured by the distance calculation portion 133. The control unit 120A estimates the number of aerosol particles from the calculated level of sound pressure of the certain speech sound on basis of a correlation. In general, a level of sound pressure detected by the detection unit 110A becomes lower as the distance between the detection unit 110A and a sound source increases. By calculating a level of sound pressure of a certain speech sound in accordance with a distance between the detection unit 110A and an utterer, therefore, the number of aerosol particles released from the utterer can be estimated more accurately.

In the aerosol quantity estimation system 100A according to the present embodiment, the detection unit 110A includes the microphones 111 and 112 that detect voices. The distance calculation portion 133 calculates a distance from the detection unit 110A to an utterer on the basis of the voices detected by the microphones 111 and 112. By detecting voices, therefore, a distance between the detection unit 110A and an utterer can be calculated, instead.

### 2-5. Modification

Although a distance from the detection unit 110A to an utterer is calculated on the basis of voices detected by the microphones 111 and 112 in the second embodiment, how the distance from the detection unit 110A to an utterer is calculated is not limited to this. The aerosol quantity estimation system 100A may also include a camera that captures an image of a voice detection range of the microphone 111 of the detection unit 110A, and the distance calculation portion 133 may calculate the distance from the detection unit 110A to an utterer using the image captured by the camera, instead. By capturing an image of the voice detection range of the detection unit 110A, therefore, the distance between the detection unit 110A and an utterer can be calculated. The camera is an example of an imager. When the distance between the detection unit 110A and an utterer is calculated using an image captured by the camera, the number of microphones included in the detection unit 110A may be one.

### Other Embodiments

Although the aerosol quantity estimation systems 100 and 100A according to the first and second embodiments may issue a warning or notify the user of the risk of infection if a calculated total value of the number of aerosol particles is larger than the certain number of aerosol particles (threshold), the present disclosure is not limited to these embodiments.

For example, if a total value is larger than the certain number of aerosol particles (threshold), the control unit 120 or 120A of the aerosol quantity estimation system 100 or 100A may control a disinfection apparatus provided in or around a space where the detection unit 110 or 110A is provided in such a way as to spray a disinfectant solution or radiate ultraviolet light in the space. The control unit 120 or 120A may control the amount of disinfectant solution sprayed or the amount of ultraviolet light radiated (ultraviolet intensity or radiation time) in accordance with a total value. The control unit 120 or 120A may control the disinfection apparatus such that the amount of disinfectant solution sprayed or the amount of ultraviolet light radiated increases as the total value increases. The disinfection apparatus is provided outside the aerosol quantity estimation system 100 or 100A. The disinfectant solution may be a liquid containing hypochlorous acid. If the number of aerosol particles with which it can be determined that the risk of infection is high is exceeded, therefore, the disinfectant solution can be sprayed or ultraviolet light can be radiated in the space. The disinfectant solution can be sprayed or ultraviolet light can be radiated in the space at appropriate times, thereby reducing the risk of infection.

If a total value is larger than the certain number of aerosol particles (threshold), for example, the control unit 120 or 120A of the aerosol quantity estimation system 100 or 100A may drive a ventilation fan or any other fan for ventilating a space where the detection unit 110 or 110A is provided. If the number of aerosol particles with which it can be determined that the risk of infection is high is exceeded, therefore, the space can be ventilated. The space can thus be effectively ventilated at appropriate times, thereby reducing the risk of infection.

The aerosol quantity estimation system 100 or 100A according to the first or second embodiment may also include a body temperature measuring unit that measures body temperature of an utterer. The control unit 120 or 120A may determine whether the body temperature measured by the body temperature measuring unit exceeds a certain temperature and, if the body temperature exceeds the certain temperature, issue a warning. A warning, therefore, can be issued if it can be determined that the risk of infection is even higher.

Although identification of monosyllables in Japanese speech sounds has been described in the first and second embodiments, the scope of the present disclosure also includes modes where monosyllables in speech sounds in another language such as English, Chinese, or French are identified. Although the recognition dictionary database 151 of phonetic symbols is used for Japanese, a word-by-word recognition dictionary database and a correlation database of the corresponding number of aerosol particles may be used for English, Chinese, or French. For Japanese, too, a word-by-word recognition dictionary database and a correlation database of the corresponding number of aerosol particles may be used instead of the recognition dictionary database 151 of phonetic symbols.

Although the aerosol quantity estimation systems according to embodiments of the present disclosure have been described, the present disclosure is not limited to these embodiments.

The processors included in the aerosol quantity estimation system according to each of the above embodiments are achieved as a large-scale integration (LSI) chip, which is typically an integrated circuit. The processors may each be achieved as a chip, or some or all of the processors may be achieved as a chip.

An integrated circuit used is not limited to an LSI chip, and a dedicated circuit or a general-purpose processor may be used, instead. A field-programmable gate array (FPGA), where an LSI chip can be programmed after fabrication thereof, or a reconfigurable processor, where connections and settings of circuit cells inside an LSI chip can be reconfigured, may be used.

In each of the above embodiments, each component may be achieved by dedicated hardware or by executing a software program suitable for the component. Each component may be achieved by reading and executing a software program stored in a storage medium such as a hard disk or a semiconductor memory using a program execution unit such as a central processing unit (CPU) or a processor, instead.

The present disclosure may be achieved as an aerosol quantity estimation method executed by the aerosol quantity estimation system or the like.

Division of the functional blocks in the block diagrams is an example, and different functional blocks may be achieved as a single block, a single functional block may be divided into different functional blocks, or some functions may be transferred to other functional blocks. A single piece of hardware or software may process similar functions of different functional blocks in parallel or a time-division manner.

Order in which the steps of each flowchart are performed is an example for specifically describing the present disclosure, and may be changed. Some of the steps may be performed simultaneously (in parallel) with another step.

Although the aerosol quantity estimation system and the aerosol quantity estimation method according to one or more aspects have been described on the basis of some embodiments, the present disclosure is not limited to these embodiments. The scope of the present disclosure also includes modes achieved by modifying the above embodiments in ways conceivable by those skilled in the art and modes constructed by combining together components from different embodiments, insofar as the spirit of the present disclosure is not deviated from.

The present disclosure also includes an infection risk evaluation system including a detection unit that detects a voice and a control unit that evaluates a risk of infection on the basis of types of speech sounds included in the voice. The detection unit in the infection risk evaluation system may have the same configuration as that of the detection unit of the aerosol quantity estimation system. The control unit of the infection risk evaluation system may have substantially the same configuration as that of the control unit of the aerosol quantity estimation system. That is, the control unit in the infection risk evaluation system estimates the number of aerosol particles released to a space where an utterer who has emitted the voice detected by the detection unit exists from certain speech sounds included in the voice on the basis of, for example, correlations between the certain speech sounds and the number of aerosol particles released from the utterer when the utterer utters the certain speech sounds. The control unit in the infection risk evaluation system can also add up the estimated number of aerosol particles and evaluate a degree of the risk of infection in accordance with a total value of the number of aerosol particles. If the risk of infection exceeds a certain degree, a warning can be issued, a disinfectant solution can be sprayed or ultraviolet light can be radiated in the space, or the space can be ventilated.

The aerosol quantity estimation system or the infection risk evaluation system in the present disclosure can be installed, for example, in an automobile. In this case, a voice detection unit of an automotive navigation system having a speech recognition function may be used as the detection unit in the aerosol quantity estimation system or the infection risk evaluation system in the present disclosure.

The present disclosure is effective as an aerosol quantity estimation system, an aerosol quantity estimation method, and the like capable of accurately estimating the number of aerosol particles caused by an utterer when the utterer has uttered speech sounds.

What is claimed is:

1. An aerosol quantity estimation system comprising:
a detector that detects a voice; and
a controller that estimates a number of aerosol particles released to a space where an utterer who has emitted the voice detected by the detector exists from a certain speech sound included in the voice on a basis of a correlation between the certain speech sound and a number of aerosol particles released from the utterer when the utterer utters the certain speech sound.

2. The aerosol quantity estimation system according to claim 1,
wherein the certain speech sound includes an utterance that uses a certain one of places of articulation used to utter speech sounds.

3. The aerosol quantity estimation system according to claim 2,
wherein the certain place of articulation includes both lips, an alveolar ridge, or a back of the alveolar ridge.

4. The aerosol quantity estimation system according to claim 1,
wherein the certain speech sound includes an utterance that uses a certain one of manners of articulation used to utter speech sounds.

5. The aerosol quantity estimation system according to claim 4,
wherein the certain manner of articulation includes a plosive, a fricative, or an affricate.

6. The aerosol quantity estimation system according to claim 1,
wherein the certain speech sound is an utterance “shi” in Japanese.

7. The aerosol quantity estimation system according to claim 1,
wherein the correlation includes a correlation between a level of sound pressure of the certain speech sound and the number of aerosol particles released from the utterer when the utterer has uttered the certain speech sound, and
wherein the controller estimates, on a basis of the correlation, the number of aerosol particles from a detected level of sound pressure, which is detected from the certain speech sound included in the voice detected by the detector.

8. The aerosol quantity estimation system according to claim 7, further comprising:
a distance calculator that calculates a distance from the detector to the utterer who has emitted the voice,
wherein the controller (i) calculates, on a basis of the detected level of sound pressure and the distance measured by the distance calculator, the level of sound pressure of the certain speech sound at a time when the utterer has uttered the certain speech sound and (ii) estimates the number of aerosol particles from the calculated level of sound pressure of the certain speech sound on the basis of the correlation.

9. The aerosol quantity estimation system according to claim 8,
wherein the detector includes microphones that detect voices, and
wherein the distance calculator calculates the distance on a basis of the voices detected by the microphones.

10. The aerosol quantity estimation system according to claim 8, further comprising:
an imager that captures an image of a voice detection range of the detector,
wherein the distance calculator calculates the distance using the image captured by the imager.

11. The aerosol quantity estimation system according to claim 1, further comprising:
a storage storing the correlation; and
a communicator communicably connected to a communication network,
wherein the controller updates the correlation stored in the storage to a new correlation between the certain speech sound and the number of aerosol particles, the new correlation being obtained by the communicator through communication with an external apparatus.

12. The aerosol quantity estimation system according to claim 1,
wherein, if the estimated number of aerosol particles exceeds a certain number of aerosol particles, the controller issues a warning.

13. The aerosol quantity estimation system according to claim 12,
wherein the controller issues the warning using an apparatus outside the aerosol quantity estimation system.

14. The aerosol quantity estimation system according to claim 13,
wherein the apparatus outside the aerosol quantity estimation system is a display apparatus provided in the space or a mobile terminal owned by the utterer.

15. The aerosol quantity estimation system according to claim 12, further comprising:
a body temperature measurer that measures body temperature of the utterer,
wherein, if the body temperature measured by the body temperature measurer exceeds a certain body temperature, the controller issues the warning.

16. The aerosol quantity estimation system according to claim 1,
wherein, if the estimated number of aerosol particles exceeds the certain number of aerosol particles, the controller sprays a disinfectant solution or radiates ultraviolet light in the space using an apparatus outside the aerosol quantity estimation system.

17. The aerosol quantity estimation system according to claim 1,
wherein, if the estimated number of aerosol particles exceeds the certain number of aerosol particles, the controller ventilates the space.

18. An aerosol quantity estimation method comprising:
detecting a voice; and
estimating a number of aerosol particles released to a space where an utterer who has emitted the detected voice exists from a certain speech sound included in the voice on a basis of a correlation between the certain speech sound and a number of aerosol particles released from the utterer when the utterer utters the certain speech sound.

19. A non-transitory computer-readable recording medium storing a program causing a computer to execute the aerosol quantity estimation method according to claim 18.

20. An aerosol quantity estimation method comprising:
detecting a voice emitted by a subject, thereby generating an audio signal; and
determining a number of aerosol particles released from the subject on a basis of (i) information indicating a relationship between a level of sound pressure of a first speech sound and a number of aerosol particles released from an utterer when the utterer utters the first speech sound and (ii) a level of sound pressure indicated by a part of the audio signal corresponding to the first speech sound.

21. An infection risk evaluation system comprising:
a detector that detects a voice; and
a controller that evaluates a risk of infection on a basis of types of speech sounds included in the voice.

22. The infection risk evaluation system according to claim 21,
wherein, if the risk of infection exceeds a certain value, the controller issues a warning.

23. The infection risk evaluation system according to claim 21,
wherein, if the risk of infection exceeds the certain value, the controller sprays a disinfection solution or radiates ultraviolet light in a space.

24. The infection risk evaluation system according to claim 21,
wherein, if the risk of infection exceeds the certain value, the controller ventilates a space.

25. A infection risk evaluation method comprising:
detecting a voice; and
evaluating a risk of infection on a basis of types of speech sounds included in the voice.
